(19) United States
(12) Patent Application Publication
(10) Pub. No.: US 2026/0193155 A1
(43) Pub. Date: Jul. 9, 2026

(54) METHODS FOR MAKING LIGHT OLEFINS BY DEHYDROGENATION THAT UTILIZE COMUBUSTION ADDITIVES THAT INCLUDE TRANSITION METALS

(71) Applicant: Dow Global Technologies LLC, Midland, MI (US)

(72) Inventors: Lin Luo, Freeport, TX (US); Yang Yang, Freeport, TX (US); Mingzhe Yu, Freeport, TX (US); Brian W. Goodfellow, Freeport, TX (US); Adrianus Koeken, Terneuzen (NL); Andrzej Malek, Midland, MI (US); Manish Sharma, Lake Jackson, TX (US)

(73) Assignee: Dow Global Technologies LLC, Midland, MI (US)

(21) Appl. No.: 18/874,666

(22) PCT Filed: Jun. 9, 2023
(86) PCT No.: PCT/US2023/068173

§ 371 Date: Dec. 13, 2024

Related U.S. Application Data
(60) Provisional application No. 63/352,013, filed on Jun. 14, 2022.

(51) Int. Cl. C07C 5/333; B01J 23/62; B01J 23/96; B01J 35/38; B01J 38/30

(52) U.S. Cl. CPC C07C 5/3337; B01J 23/62; B01J 23/96; B01J 35/38; B01J 38/30; C07C 2523/08; C07C 2523/42

(57) ABSTRACT

A method for making light olefins by dehydrogenation may include operating a catalytic dehydrogenation process, monitoring a composition of a combustion gas in the combustor to detect a concentration of one or more hydrocarbons, and selectively adding a combustion additive with the catalyst when the combustion gas comprises one or more hydrocarbons in an amount greater than 5% of a lower flammability level of the combustion gas at a temperature and pressure of the combustor. A Jet cup attrition index of the combustion additive may be greater than a Jet cup attrition index of the catalyst. The combustion additive may comprise from 1 wt. % to 10 wt. % of one or more transition metals exclusive of gallium and noble metals, from 0 parts per million by weight (ppmw) to 100 ppmw of gallium and noble metals, and at least 85 wt. % support.

### CROSS-REFERENCE TO RELATED APPLICATION

This application claims the benefit of U.S. Provisional Application Ser. No. 63/352,013 filed Jun. 14, 2022, the entire disclosure of which is hereby incorporated herein by reference.

### TECHNICAL FIELD

Embodiments described herein generally relate to chemical processing and, more specifically, to methods and systems for light olefin production.

### BACKGROUND

Light olefins, such as propylene, may be used as base materials to produce many different materials, such as polypropylene, isopropanol, and acrylic acid, which may be used in, e.g., packaging, construction, and textiles. As a result of this utility, there is a worldwide demand for light olefins. Suitable processes for producing light olefins generally depend on the given chemical feed and include those that utilize fluidized catalysts. For example, light olefins may be formed by the catalytic dehydrogenation of alkanes in a fluidized bed reactor. However, there is a need for improvement in the systems and associated catalysts used to make light olefins.

### SUMMARY

Methods and associated systems for making light olefins by dehydrogenation can include reacting hydrocarbon-containing feeds over a catalyst in a reactor. Following the endothermic dehydrogenation reaction, the catalyst can be passed to a combustor where it is heated by combustion of a supplemental fuel. The catalyst provides both dehydrogenation activity in the reactor and combustion activity in the combustor for supplemental fuel combustion. Sometimes, a combustion rate may be below that which is desired, while dehydrogenation rate is sufficient. For example, in some embodiments, it has been found that, over time, the catalytic activity of the catalyst is reduced for combustion more than for dehydrogenation. In other embodiments, process fluctuations such as the composition of the supplemental fuel may require additional catalytic activity for combustion. In some conventional systems, fresh catalyst is added to the system to compensate for the loss in performance due to catalyst aging and/or catalyst attrition in order to maintain acceptable dehydrogenation activity and/or to maintain acceptable supplemental fuel combustion activity. Loss of catalyst combustion activity may limit usable fuel compositions, which may negatively impact process economics or flexibility.

The catalyst systems and methods for producing olefins of the present disclosure may efficiently maintain dehydrogenation catalytic activity in the reactor and maintain sufficient combustion activity in the combustor of the system. In one or more embodiments, this is accomplished, at least in part, by the utilization of both a catalyst and a combustion additive that is selectively added to the process when combustion activity is lesser than desired. The combustion additive can include less than 100 ppmw gallium and noble metals used for dehydrogenation activity, thereby reducing the economic cost of the material. However, the combustion additive can selectively promote combustion activity when desired. Further, the combustion additive can have a greater attrition rate than the catalyst and, therefore, allow for removal of the combustion additive from the system separate from removal of catalyst. In some embodiments, the different attrition properties of the catalyst and combustion additive can reduce the dilution of the catalyst in the system, thereby improving the catalytic activity of the catalyst while reducing the economic cost of the process.

According to one or more embodiments of the present disclosure, a method for making light olefins by dehydrogenation may comprise operating a catalytic dehydrogenation process, monitoring a composition of a combustion gas in the combustor to detect a concentration of one or more hydrocarbons, and selectively adding a combustion additive with the catalyst when the combustion gas comprises one or more hydrocarbons in an amount greater than 5% of a lower flammability level of the combustion gas at a temperature and pressure of the combustor. The operating may comprise contacting a hydrocarbon-containing feed with a catalyst in a reactor to form an olefin-containing effluent, at least partially separating the olefin-containing effluent from the catalyst, passing the catalyst to a combustor and heating the catalyst by combusting a supplemental fuel, wherein the supplemental fuel comprises methane in an amount of greater than or equal to 1 mol. %, and passing the catalyst from the combustor to the reactor, such that at least a portion of the catalyst continuously cycles between the reactor and the combustor. A Jet cup attrition index of the combustion additive is greater than a Jet cup attrition index of the catalyst, where the Jet cup attrition is measured with 45 μm threshold after 6 hours at ambient temperature, 300 ft/s jet velocity, a flow rate of 98 L/min, and a sample loading of 100 grams. The combustion additive comprises from 1 wt. % to 10 wt. % of one or more transition metals exclusive of gallium and noble metals, from 0 parts per million by weight (ppmw) to 100 ppmw of gallium and noble metals, and at least 85 wt. % support.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawings and claims, or recognized by practicing the described embodiments. The drawings are included to provide a further understanding of the embodiments and, together with the detailed description, serves to explain the principles and operations of the claimed subject matter. However, the embodiments depicted in the drawings are illustrative and exemplary in nature, and not intended to limit the claimed subject matter.

### BRIEF DESCRIPTION OF THE DRAWINGS

The following detailed description may be better understood when read in conjunction with the following drawings, in which:

FIG. 1 schematically depicts a reactor system, according to one or more embodiments of the present disclosure; and

FIG. 2 is a flowchart of a method, according to one or more embodiments of the present disclosure.

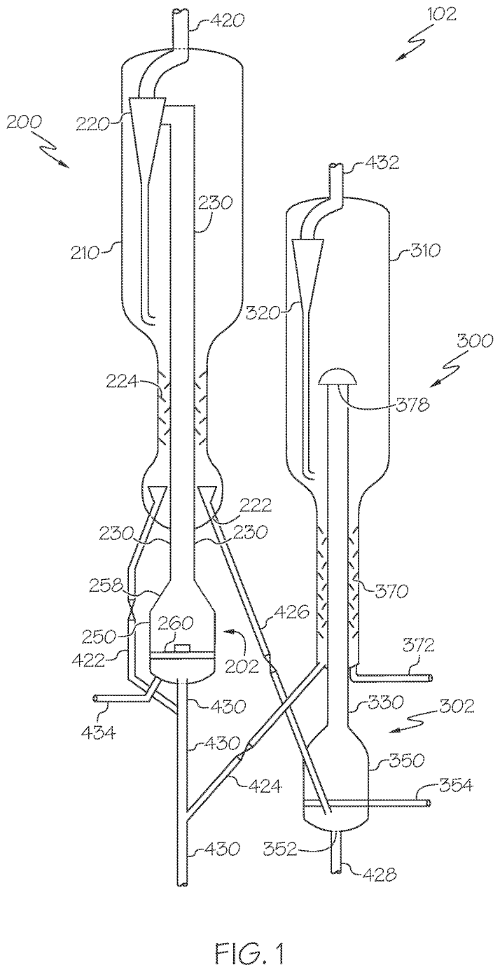

When describing the simplified schematic illustration of FIG. 1, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Reference will now be made in greater detail to various embodiments, some of which are illustrated in the accompanying drawings.

### DETAILED DESCRIPTION

The present disclosure is directed to methods for making light olefins by dehydrogenation where a combustion additive is utilized. The methods may generally include operating a catalytic dehydrogenation process, monitoring a composition of a combustion gas in the combustor to detect a concentration of one or more hydrocarbons, and selectively adding a combustion additive with the catalyst when the combustion gas comprises one or more hydrocarbons in an amount greater than 5% of a lower flammability level of the combustion gas at a temperature and pressure of the combustor. In embodiments described herein, the combustion additive includes from 1 wt. % to 10 wt. % of one or more transition metals exclusive of gallium and noble metals, from 0 ppmw to 100 ppmw of gallium and noble metals, and at least 85 wt. % support. According to some embodiments, such combustion additives may be particularly well suited for fluidized dehydrogenation of light alkanes to light olefins, such as propane to propylene, where a supplemental fuel such as methane is used to heat the catalyst.

Embodiments presently disclosed are described in detail herein in the context of the reactor system of FIG. 1 operating as a fluidized dehydrogenation reactor system to produce light olefins, such as propylene. However, it should be understood that the principles disclosed and taught herein may be applicable to other systems which utilize different system components oriented in different ways, or different reaction schemes utilizing various catalyst compositions. For example, the concepts described may be equally applied to other systems with alternate reactor units and regeneration units, such as those that operate under non-fluidized conditions or include downers rather than risers. It should be further understood that not all portions of FIG. 1 should be construed as essential to the claimed subject matter.

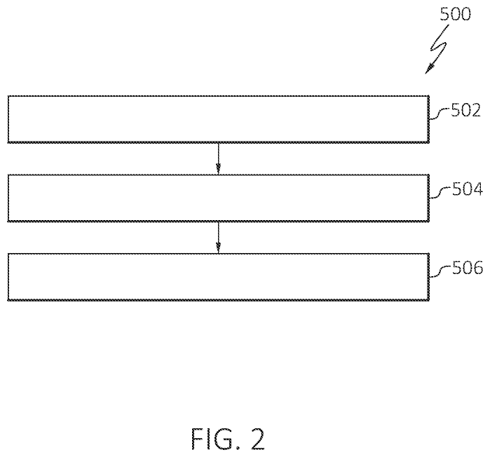

Now referring to FIG. 2, a flow chart depicting a method 500 for making light olefins by dehydrogenation is depicted, according to one or more embodiments described herein. Step 502 generally includes operating a catalytic dehydrogenation process, step 504 includes monitoring a composition of a combustion gas in the combustor to detect a concentration of one or more hydrocarbons, and step 506 includes selectively adding a combustion additive with the catalyst when the combustion gas comprises one or more hydrocarbons in an amount greater than 5% of a lower flammability level of the combustion gas at a temperature and pressure of the combustor.

In one or more embodiments, the operating of the dehydrogenation process in step 502 generally includes contacting a hydrocarbon-containing feed with a catalyst in a reactor to form an olefin-containing effluent, wherein coke forms on the catalyst in the reactor; and at least partially separating the olefin-containing effluent from the catalyst. Step 502 may further include passing the catalyst to a combustor and heating the catalyst by combusting a supplemental fuel and at least a portion of the coke on the catalyst, wherein the supplemental fuel comprises methane in an amount of greater than or equal to 1 mol. %, and passing the catalyst from the combustor to the reactor, such that at least a portion of the catalyst continuously cycles between the reactor and the combustor. Such embodiments are described hereinafter in the context of the system of FIG. 1.

Now referring to FIG. 1, an example reactor system 102 that may be suitable for use with the methods and/or apparatuses described herein is schematically depicted. The reactor system 102 generally comprises multiple system components, such as a reactor portion 200 and a catalyst processing portion 300. As described herein, “system components” refer to portions of the reactor system 102, such as reactors, separators, transfer lines, combinations thereof, and the like. As used herein in the context of FIG. 1, the reactor portion 200 generally refers to the portion of a reactor system 102 in which the major process reaction takes place (e.g., dehydrogenation) to form the product stream. A feed stream enters the reactor portion 200, is converted to a product stream (containing product and unreacted feed), and exits the reactor portion 200. The reactor portion 200 comprises a reactor 202 which may include an upstream reactor section 250 and a downstream reactor section 230. According to one or more embodiments, as depicted in FIG. 1, the reactor portion 200 may additionally include a catalyst separation section 210, which serves to separate the catalyst from the chemical products formed in the reactor 202. Also, as used herein, the catalyst processing portion 300 generally refers to the portion of the reactor system 102 where the catalyst is in some way processed, such as by combustion, to, e.g., improve catalytic activity by decoking and/or heat the catalyst. The catalyst processing portion 300 may comprise a combustor 350 and a riser 330, and may additionally comprise a catalyst separation section 310. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430).

Generally as is described herein, in embodiments illustrated in FIG. 1, catalyst is cycled between the reactor portion 200 and the catalyst processing portion 300. It should be understood that when “catalysts” are referred to herein, they may refer to solid materials that are catalytically active for a desired reaction, or may equally refer to other particulate solids referenced with respect to the system of FIG. 1 which do not necessarily have catalytic activity but affect the reaction, such as oxygen carriers. The terms “catalytic activity” and “catalyst activity” refer to the degree to which the catalyst is able to catalyze the reactions conducted in the reactor system. The catalyst that exits the reactor portion 200 may be deactivated catalyst. As used herein, “deactivated” may refer to a catalyst which has reduced catalytic activity or is cooler as compared to catalyst entering the reactor portion 200. However, deactivated catalyst may maintain some catalytic activity. Reduced catalytic activity may result from contamination with a substance such as coke. Reactivation (sometimes called “regeneration” herein) may remove the contaminant such as coke, raise the temperature of the catalyst, or both. In embodiments, deactivated catalyst may be reactivated by catalyst reactivation in the catalyst processing portion 300. The deactivated catalyst may be reactivated by, but not limited to, removing coke by combustion, recovering catalyst acidity, oxidizing the catalyst, other reactivation process, or combinations thereof. In some embodiments, the catalyst may be heated during reactivation by combustion of a supplemental fuel, such as methane, ethane, hydrogen, propane, natural gas, or combinations thereof. The reactivated catalyst from the catalyst processing portion 300 is then passed back to the reactor portion 200.

In embodiments, additional fresh catalyst can be added to the reactor system 102 to compensate for reduced catalytic activity of the catalyst. The catalyst may exhibit reduced catalytic activity due to catalyst loss from attrition or performance decay over time.

In non-limiting examples, the reactor system 102 described herein may be utilized to produce light olefins from hydrocarbon-containing feeds. According to one or more embodiments, the reaction may be a dehydrogenation reaction. According to such embodiments, the hydrocarbon-containing feed may comprise one or more of ethane, propane, n-butane, and i-butane. In one or more embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon-containing feed may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

In one or more embodiments, the catalyst may comprise, consist essentially of, or consist of one or more of gallium, indium, or thallium; one or more of platinum, palladium, rhodium, iridium, ruthenium, or osmium; and a support. As described herein, “consisting essentially of” refers to materials with less than 1 wt. % of the non-recited materials (i.e., consisting essentially of A and B means A and B combined are at least 99 wt. % of the composition). As is described herein, the catalyst may be solid particles suitable for fluidization.

In one or more embodiments, the catalyst may comprise one or more of gallium, indium, or thallium in an amount of from 0.05 wt. % to 10 wt. % based on the total mass of the catalyst. For example, the catalyst may comprise one or more of gallium, indium, or thallium in an amount from 0.05 wt. % to 0.1 wt. %, from 0.1 wt. % to 0.25 wt. %, from 0.25 wt. % to 0.5 wt. %, from 0.5 wt. % to 0.75 wt. %, from 0.75 wt. % to 1 wt. %, from 1 wt. % to 2 wt. %, from 2 wt. % to 3 wt. %, from 3 wt. % to 4 wt. %, from 4 wt. % to 5 wt. %, from 5 wt. % to 6 wt. %, from 6 wt. % to 7 wt. %, from 7 wt. % to 8 wt. %, from 8 wt. % to 9 wt. %, from 9 wt. % to 10 wt. %, or any combination of these ranges. In some embodiments, the catalyst may comprise one or more of gallium, indium, or thallium in an amount from 0.05 wt. % to 5 wt. %, from 0.05 wt. % to 4 wt. %, or from 0.05 wt. % to 3 wt. %. In some embodiments, the catalyst comprises only gallium but not indium or thallium, only indium but not gallium or thallium, or only thallium but not gallium or indium. It should be understood that the compositional ranges describing the amount of gallium, indium, and thallium represent ranges for any one of these materials, or for the combination of these materials. Without being bound by theory, it is believed that compositions having one or more of gallium, indium, or thallium in an amount less than 0.05 wt. % negatively impacts the catalyst's ability to catalyze the alkane dehydrogenation process by lowering both the percentage of total alkane dehydrogenated and the percentage of dehydrogenated alkane that is the intended product. However, it is believed that compositions having one or more of gallium, indium, or thallium in an amount exceeding 10 wt. % may increase the economic cost of operation while not providing substantial improvement in the catalytic function catalyst.

In one or more embodiments, the catalyst may comprise one or more of platinum, palladium, rhodium, iridium, ruthenium, or osmium in an amount from 5 ppmw to 1000 ppmw based on the total mass of the catalyst. For example, the catalyst may comprise one or more of platinum, palladium, rhodium, iridium, ruthenium, or osmium in an amount from 5 ppmw to 50 ppmw, from 50 ppmw to 100 ppmw, from 100 ppmw to 200 ppmw, from 200 ppmw to 300 ppmw, from 300 ppmw to 400 ppmw, from 400 ppmw to 500 ppmw, from 500 ppmw to 600 ppmw, from 600 ppmw to 700 ppmw, from 700 ppmw to 800 ppmw, from 800 ppmw to 900 ppmw, from 900 ppmw to 1000 ppmw, or any combination of these ranges. In some embodiments, the catalyst may comprise one or more of platinum, palladium, rhodium, iridium, ruthenium, or osmium in an amount from 5 ppmw to 900 ppmw, from 5 ppmw to 800 ppmw, from 5 ppmw to 600 ppmw, from 5 ppmw to 500 ppmw, or from 10 ppmw to 400 ppmw. In some embodiments, the catalyst comprises only platinum but not palladium, rhodium, iridium, ruthenium, or osmium, only palladium but not platinum, rhodium, iridium, ruthenium, or osmium, only rhodium, but not platinum palladium, iridium, ruthenium, or osmium, only iridium, but not platinum palladium, rhodium, ruthenium, or osmium, only ruthenium but not platinum, palladium, rhodium, iridium, or osmium, or only osmium but not platinum, palladium, rhodium, iridium, or ruthenium. It should be understood that the compositional ranges describing the amount of platinum, palladium, rhodium, iridium, ruthenium, and osmium represent ranges for any one of these materials, or for the combination of these materials. Without being bound by theory, it is believed that compositions having one or more of platinum, palladium, rhodium, iridium, ruthenium, or osmium in an amount less than 5 ppmw negatively impacts the catalyst's ability to catalyze the alkane dehydrogenation process by lowering both the percentage of total alkane dehydrogenated and the percentage of dehydrogenated alkane that is the intended product.

As is described herein, in one or more embodiments, the catalyst may comprise a support. The support may comprise one or more of alumina, silica-containing alumina, zirconia-containing alumina, or titania-containing alumina. The support may be present in an amount of at least 50 wt. % relative to the total weight of the catalyst, such as at least 55 wt. %, at least 60 wt. %, at least 65 wt. %, at least 70 wt. %, at least 75 wt. %, at least 80 wt. %, or even at least 85 wt. %. In some embodiments, the support comprises less than or equal to 95 wt. % of the catalyst. Generally, the wt. % of the support may fill the remainder of the total catalyst not specified by other materials.

In one or more embodiments, the catalyst may optionally comprise one or more alkali metals, one or more alkaline earth metals, or both in an amount from 0.01 wt. % to 1 wt. % based on the total weight of the catalyst. For example, the catalyst may comprise one or more alkali metals, one or more alkaline earth metals, or both in an amount from 0.01 wt. % to 0.05 wt. %, form 0.05 wt. % to 0.1 wt. %, from 0.1 wt. % to 0.2 wt. %, from 0.2 wt. % to 0.3 wt. %, from 0.3 wt. % to 0.4 wt. %, from 0.4 wt. % to 0.5 wt. %, from 0.5 wt. % to 0.6 wt. %, from 0.6 wt. % to 0.7 wt. %, from 0.7 wt. % to 0.8 wt. %, from 0.8 wt. % to 0.9 wt. %, from 0.9 wt. % to 1 wt. %, or any combination of these ranges. In some embodiments, the catalyst may comprise one or more alkali metals, one or more alkaline earth metals, or both from 0.01 wt. % to 0.75 wt. %, from 0.02 wt. % to 0.6 wt. %, from 0.03 wt. % to 0.5 wt. %, from 0.04 wt. % to 0.4 wt. %, or from 0.05 wt. % to 0.3 wt. %.

In one or more embodiments, the catalyst may include solid particulates that are capable of fluidization. In some embodiments, the catalyst may exhibit properties known in the industry as “Geldart A” or “Geldart B” properties. Catalyst type may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7 (1973) 285-292, the disclosures of which are incorporated herein by reference in their entireties.

Geldart Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U-Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal [symbol] ; or as the <45 micrometers (μm) proportion is increased; or as pressure, temperature, viscosity, and density of the gas increase. In general, the particles may exhibit a small mean particle size and/or a particle density (ρp) of ρp<1.4 grams per cubic centimeter (g/cm³), fluidize easily with smooth fluidization at low gas velocities, and may exhibit controlled bubbling with small bubbles at higher gas velocities. As used herein, the term “particle density (ρp)” refers to the envelope density, which includes the pore spaces within the material particle in the volume measurement (as determined using ASTM D4284-12), but excludes the interparticle volume.

Geldart Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U-Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, the particles may exhibit a mean particle size ( [symbol] ) of 40 μm< [symbol] <500 μm and a particle density (ρp) is 1.4<ρp<4 g/cm³.

In one or more embodiments, the catalyst may be prepared via incipient wetness impregnation also known as dry impregnation or capillary impregnation. For example, such a process is described in Marceau et al., Impregnation and Drying, Synthesis of Solid Catalysts 59 (2008), which is incorporated herein by reference in its entirety. For example, the support may be impregnated using nitrate or amine nitrate metal precursors, then dried at temperatures less than 200° C., and then calcined at temperatures less than 800° C. to produce the catalyst. For example, in some embodiments, the method of making the catalyst may comprise impregnating the support with gallium, and platinum; drying the support; and calcining the support, wherein the catalyst comprises from 0.05 wt. % to 10 wt. % of gallium, from 5 ppmw to 1000 ppmw of platinum, and at least 85 wt. % support.

Incipient wetness sequential impregnation allows the support to be impregnated with metals in a sequential order where some metals may be impregnated onto the support before others. The order of impregnation can therefore be altered as desired. Additionally, other suitable methods for making the catalysts described herein are contemplated, as would be known by those skilled in the art.

As described with respect to FIG. 1, the feed stream may enter feed inlet 434 into the reactor 202, and the product stream may exit the reactor system 102 via pipe 420. According to one or more embodiments, the reactor system 102 may be operated by feeding a chemical feed (e.g., in a feed stream) and a fluidized catalyst into the upstream reactor section 250. The chemical feed contacts the catalyst in the upstream reactor section 250, and each flow upwardly into and through the downstream reactor section 230 to produce a chemical product.

Now referring to FIG. 1 in detail, the reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430, which, in operation may provide reactivated catalyst in a feed stream to the reactor portion 200. The reactivated catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250, where in such embodiments some of the catalyst is not passed through the catalyst processing portion 300. The catalyst can also be fed via standpipe 422 directly to the upstream reactor section 250 (not depicted in FIG. 1). This catalyst may be somewhat deactivated, but may still, in some embodiments, be suitable for reaction in the upstream reactor section 250, particularly when used in combination with reactivated catalyst.

Still referring to FIG. 1, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 250 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating above choking velocity.

According to embodiments, the chemical product and the catalyst may be passed out of the downstream reactor section 230 to a separation device 220 in the catalyst separation section 210, where the catalyst is separated from the chemical product, which is transported out of the catalyst separation section 210. According to one or more embodiments, following separation from vapors in the separation device 220, the catalyst may generally move through a stripper 224 to the catalyst outlet port 222 where the catalyst is transferred out of the reactor portion 200 via standpipe 426 and into the catalyst processing portion 300.

According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the invention.

Still referring to FIG. 1, the separated catalyst is passed from the catalyst separation section 210 to the combustor 350. In the combustor 350, the catalyst may be processed by, for example, combustion with oxygen. For example, and without limitation, the catalyst may be de-coked and/or supplemental fuel may be combusted to heat the catalyst. The catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel, referred to herein as flue gas). The flue gas may pass out of the catalyst processing portion 300 via outlet pipe 432. The separated catalyst is then passed through the oxygen treatment zone 370 within the catalyst separation section 310 to the upstream reactor section 250 via standpipe 424 and transport riser 430, where it is further utilized in a catalytic reaction. Thus, the catalyst, in operation, may cycle between the reactor portion 200 and the catalyst processing portion 300. In general, the processed chemical streams, including the feed streams and product streams may be gaseous, and the catalyst may be fluidized particulate solid.

Referring now to the catalyst processing portion 300, as depicted in FIG. 1, the combustor 350 of the catalyst processing portion 300 may include one or more lower reactor portion inlet ports 352 and may be in fluid communication with the riser 330. Oxygen-containing gas, such as air, may be passed through pipe 428 into the combustor 350. The combustor 350 may be in fluid communication with the catalyst separation section 210 via standpipe 426, which may supply spent catalyst from the reactor portion 200 to the catalyst processing portion 300 for regeneration. The combustor 350 and riser 330, collectively referred to as the catalyst combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream, to the combustor 350.

As described in one or more embodiments, following separation of flue gas from catalyst in the riser termination separator 378 and secondary separation device 320, treatment of the processed catalyst with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040. The fluidization regime within the oxygen treatment zone may be bubbling bed type fluidization. The oxygen treatment zone 370 may include an oxygen-containing gas inlet 372, which may supply an oxygen-containing gas to the oxygen treatment zone 370 for oxygen treatment of the catalyst.

In one or more embodiments, the light olefins may be present in a “product stream” sometimes called an “olefin-containing effluent” and include light olefins. Such a stream exits the reactor system of FIG. 1 and may be subsequently processed. As used in the present disclosure, the term “light olefins” refers to one or more of ethylene, propylene, and butene. The term butene includes any isomers of butene, such as α-butylene, cis-β-butylene, trans-β-butylene, and isobutylene. In some embodiments, the olefin-containing effluent includes at least 20 wt. % light olefins based on the total weight of the olefin-containing effluent. For example, the olefin-containing effluent may include at least 25 wt. % light olefins, at least 30 wt. % light olefins, at least 35 wt. % light olefins, at least 40 wt. % light olefins, at least 45 wt. % light olefins, at least 50 wt. % light olefins, at least 55 wt. % light olefins, at least 60 wt. % light olefins, or at least 65 wt. % light olefins based on the total weight of the olefin-containing effluent. The olefin-containing effluent may further comprise unreacted components of the feed stream, as well as other reaction products that are not considered light olefins. The light olefins may be separated from unreacted components in subsequent separation steps.

Now referring again to FIG. 2, step 504 generally includes monitoring a composition of a combustion gas in the combustor to detect a concentration of one or more hydrocarbons. The monitoring can be used to detect when a combustion activity in the combustor is less than a desired threshold activity. As described herein, “combustion activity” refers to the rate of chemical combustion. Generally, combustion activity may be monitored by the temperature of the catalyst leaving the combustor. In other embodiments, the combustion activity can be monitored by on-line analysis of a composition of flue gas.

Without being bound by theory, it is believed that several circumstances may lead to reduced combustion activity. In one or more embodiments, catalyst activity is degraded over time and combustion catalytic activity is reduced more so than dehydrogenation activity. In such embodiments, the addition of the combustion additive may allow for normal operation without the need for the addition of fresh catalyst, since dehydrogenation activity is not reduced to an amount where fresh catalyst is needed and the combustion can be enhanced by use of the combustion additive.

In embodiments, the composition of the supplemental fuel may be changed, which may result in a change in the combustion rate of the supplemental fuel. For example, methane may combust at a lesser rate than other fuels such as hydrogen, propane, etc. In such embodiments, an increase in the amount of the combustion additive in the reactor system may raise the combustion rate to an acceptable level. Moreover, the combustion additives described herein may, in some embodiments, provide enhanced combustion activity for the combustion of methane.

Still referring to FIG. 2, step 506 generally includes selectively adding a combustion additive with the catalyst when the combustion gas comprises one or more hydrocarbons in an amount greater than 5% of a lower flammability level of the combustion gas at a temperature and pressure of the combustor. In one or more embodiments, the combustion additive may be added with the catalyst in the reactor system 102 when the combustion gases (i.e., the gases produced by combusting the combustion fuel in the combustor 350) comprise one or more hydrocarbons (e.g., methane, ethane, and/or propane) in an amount greater than 5% of a lower flammability limit (LFL) of the combustion gases at a temperature and pressure of the catalyst-processing portion 300, such as the combustor 350. For example, the combustion additive may be added with the catalyst to the reactor system 102 when the combustion gases comprise one or more hydrocarbons in an amount greater than 10% of the LFL of the combustion gases at a temperature and pressure of the catalyst-processing portion 300. As used in the present disclosure, the term “lower flammability limit” refers to the lower end of the concentration range over which a flammable mixture of gas or vapor in air can be ignited at a given temperature and pressure. The LFL of the combustion gases may be determined by reactive chemistry testing or as described by Michael G. Zabetakis, Flammability Characteristics of Combustible Gases and Vapors, 627 BUREAU OF MINES 1 (1965), with pressure adjustments according to Coward et al., Limits of Flammability of Gases and Vapors, 503 BUREAU OF MINES 1 (1952).

In one or more embodiments, the combustion additive can comprise one or more transition metals. As used herein, the phrase “transition metals” refers to metals selected from elements of IUPAC groups 3-12. In one or more embodiments, the combustion additive can comprise manganese, iron, cobalt, tin, or combinations of two or more thereof. In one or more embodiments, the combustion additive can be selected from manganese, iron, cobalt, tin, and combinations of two or more thereof.

In one or more embodiments, the combustion additive can comprise from 1 wt. % to 10 wt. % of one or more transition metals exclusive of gallium and noble metals based on the total weight of the combustion additive. For instance, the combustion additive can comprise one or more transition metals exclusive of gallium and noble metals in an amount from 1 wt. % to 2 wt. %, from 1 wt. % to 3 wt. %, from 1 wt. % to 4 wt. %, from 1 wt. % to 5 wt. %, from 1 wt. % to 6 wt. %, from 1 wt. % to 7 wt. %, from 1 wt. % to 8 wt. %, from 1 wt. % to 9 wt. %, from 1 wt. % to 10 wt. %, from 2 wt. % to 3 wt. %, from 2 wt. % to 4 wt. %, from 2 wt. % to 5 wt. %, from 2 wt. % to 6 wt. %, from 2 wt. % to 7 wt. %, from 2 wt. % to 8 wt. %, from 2 wt. % to 9 wt. %, or from 2 wt. % to 10 wt. %, based on the total weight of the combustion additive. Without intending to be bound by any particular theory, it is believed that combustion additives having one or more transition metals in an amount less than 1 wt. % may require a higher amount of additive used in the system to achieve the catalytic activity desired. However, it is believed that combustion additives having one or more transition metals in an amount exceeding 10 wt. % may have reduced efficiency of combustion performance.

In one or more embodiments, the combustion additive may comprise, consist essentially of, or consist of one or more of manganese, iron, cobalt, or tin in an amount from 1 wt. % to 10 wt. % based on the total weight of the combustion additive. For instance, the combustion additive can comprise one or more of manganese, iron, cobalt, or tin in an amount from 1 wt. % to 2 wt. %, from 1 wt. % to 3 wt. %, from 1 wt. % to 4 wt. %, from 1 wt. % to 5 wt. %, from 1 wt. % to 6 wt. %, from 1 wt. % to 7 wt. %, from 1 wt. % to 8 wt. %, from 1 wt. % to 9 wt. %, from 1 wt. % to 10 wt. %, from 2 wt. % to 3 wt. %, from 2 wt. % to 4 wt. %, from 2 wt. % to 5 wt. %, from 2 wt. % to 6 wt. %, from 2 wt. % to 7 wt. %, from 2 wt. % to 8 wt. %, from 2 wt. % to 9 wt. %, or from 2 wt. % to 10 wt. % based on the total weight of the combustion additive. In some embodiments, the combustion additive comprises only manganese but not iron, cobalt or tin, only iron, but not manganese, cobalt, or tin, only cobalt, but not manganese, iron, or tin, or only tin, but not manganese, iron, or cobalt. It should be understood that the compositional ranges describing the amount manganese, iron, cobalt, and tin represent ranges for any one of these materials, or for the combination of these materials.

In one or more embodiments, the combustion additive can comprise less than 100 ppmw gallium and noble metals based on the total weight of the combustion additive. As used herein, “noble metals” refer to ruthenium, rhodium, palladium, osmium, iridium, platinum, silver, and gold. In one or more embodiments the combustion additive does not comprise gallium and noble metals. In one or more embodiments, the combustion additive can comprise an amount of gallium and noble metals from 0 ppmw to 100 ppmw, from 0 ppmw to 50 ppmw, from 0 ppmw to 25 ppmw, from 0 ppmw to 10 ppmw, or from 0 ppmw to 5 ppmw based on the total weight of the combustion additive. Without being bound by any particular theory, it is believed that the exclusion or reduction of gallium and noble metals in the combustion additive can reduce the economic costs of the dehydrogenation process.

In one or more embodiments, the combustion additive can comprise less than 5 wt. % one or more alkali metals, one or more alkaline earth metals, or both based on the total weight of the combustion additive. For example, the combustion additive can comprise from 0 wt. % to 5 wt. %, from 0 wt. % to 4 wt. %, from 0 wt. % to 3 wt. %, from 0 wt. % to 2 wt. %, from 0 wt. % to 1 wt. %, from 1 wt. % to 5 wt. %, from 1 wt. % to 4 wt. %, from 1 wt. % to 3 wt. %, from 1 wt. % to 2 wt. %, from 2 wt. % to 5 wt. %, from 2 wt. % to 4 wt. %, from 2 wt. % to 3 wt. %, from 3 wt. % to 5 wt. %, from 3 wt. % to 4 wt. %, or from 4 wt. % to 5 wt. % one or more alkali metals, one or more alkaline earth metals, or both based on the total weight of the combustion additive. Without intending to be bound by any particular theory, it is believed that combustion additives having alkali metals, alkaline earth metals, or both can reduce secondary reaction of desired dehydrogenated products. However, it is believed that combustion additives having alkali metals, alkaline earth metals, or both in an amount exceeding 5 wt. % may no longer provide such function.

In one or more embodiments, the combustion additive can include a support material. Specifically, the combustion additive may include a transition metal, alkali metal, and/or alkaline earth metal disposed and/or dispersed on a support. In some embodiments, the support material includes one or more of alumina, silica, titanium oxide, and zirconium. For example, the support material may include one or more of alumina, silica-containing alumina, titanium oxide-containing alumina, and zirconium-containing alumina. The support may be present in an amount of at least 85 wt. % relative to the total weight of the combustion additive. In some embodiments, the support comprises less than or equal to 99 wt. % of the combustion additive. Generally, the wt. % of the support may fill the remainder of the total combustion additive not specified by other materials.

In one or more embodiments, the combustion additive may be prepared via incipient wetness impregnation also known as dry impregnation or capillary impregnation. For example, such a process is described in Marceau et al., Impregnation and Drying, Synthesis of Solid Catalysts 59 (2008), which is incorporated herein by reference in its entirety. For example, the support may be impregnated using nitrate or amine nitrate metal precursors, then dried at temperatures less than 200° C., and then calcined at temperatures less than 800° C. to produce the combustion additive. For example, in some embodiments, the method of making the combustion additive may comprise impregnating the support with a transition metal; drying the support; and calcining the support, wherein the combustion additive comprises from 1 wt. % to 10 wt. % of one or more transition metals exclusive of gallium and noble metals, from 0 ppmw to 100 ppwm of gallium and noble metals, and at least 85 wt. % support.

Incipient wetness sequential impregnation allows the support to be impregnated with metals in a sequential order where some metals may be impregnated onto the support before others. The order of impregnation can be therefore be altered as desired. Additionally, other suitable methods for making the combustion additives described herein are contemplated, as would be known by those skilled in the art.

In one or more embodiments, a dehydrogenation catalytic activity of the combustion additive can be less than 25% of a dehydrogenation catalytic activity of the catalyst, where the dehydrogenation catalytic activity refers to the feed conversion percent in the propane dehydrogenation test described herein. For instance, the dehydrogenation catalytic activity of the combustion additive can be less than 20%, less than 15%, less than 10%, or even less than 5% of the dehydrogenation catalytic activity of the catalyst. In embodiments, the dehydrogenation catalytic activity is a propane dehydrogenation catalytic activity.

In one or more embodiments, an approach to equilibrium (ATE) conversion of the combustion additive can be less than 15% of an ATE conversion of the catalyst, where the ATE conversion refers to the approach to equilibrium conversion calculation in the test methods described herein. For instance, the approach to equilibrium (ATE) conversion of the combustion additive can be less than 12%, less than 10%, less than 8%, less than 7%, less than 6%, or even less than 5% of the ATE conversion of the catalyst as measured in the approach to equilibrium conversion calculation in the test methods described herein.

In embodiments, addition of the combustion additive with the catalyst can result in dilution of the catalyst, thereby reducing the dehydrogenation catalytic activity of the system. However, the attrition properties of the combustion additive and the catalyst can be controlled to reduce or eliminate this dilution effect. During operation of the catalytic dehydrogenation process, both the catalyst and the combustion additive can undergo attrition. As used herein, the term “attrition” can refer to the unwanted mechanical breakage and degradation of solid materials via abrasion and fracturing mechanisms, such as the catalyst and combustion additive, into catalyst fines and smaller particles. Attrition of the catalyst can result in catalyst loss from the unit as fines with particle size below a certain threshold, such as less than 20 microns, may not be retained by the reactor system 102. Attrition of the combustion additive can result in decreased combustion activity. Attrition of the combustion additive can also reduce the dilution effect on dehydrogenation activity. As solids undergo attrition, the attrited materials can be passed out of the reactor system. The attrition properties of the catalysts and combustion additives can affect the lifetime of the catalysts and combustion additives in fluidized bed processes, such as the fluidized dehydrogenation reactor described herein. For example, if the combustion additive has a higher attrition rate than the catalyst in commercial operation, the combustion additive can stay in the unit for a period of time that is shorter than the catalyst in the fluidized bed process. Attrition evaluation can be carried out by various lab-scale or pilot-scale attrition tests, such as ASTM D5757, submerged-jet test, and jet cup attrition test.

Jet cup attrition index is one of the standard lab testing methods to characterize catalyst attrition property for fluidized bed applications, as described in Cocco et al., Jet Cup Attrition Testing, 200 Powder Technology 224 (2010). Although lab testing cannot provide actual attrition rate in a commercial scale unit, the lab testing provides ranks of attrition tendency. In one or more embodiments, a Jet cup attrition index of the combustion additive is greater than a Jet cup attrition index of the catalyst, where the Jet cup attrition is measured with 45 μm threshold after 6 hours at ambient temperature, 300 ft/s jet velocity, a flow rate of 98 L/min, and a sample loading of 100 grams. In embodiments, a Jet cup attrition index of the combustion additive is at least 4 times that of a Jet cup attrition index of the catalyst, where the Jet cup attrition is measured with 45 μm threshold after 6 hours at ambient temperature, 300 ft/s jet velocity, a flow rate of 98 L/min, and a sample loading of 100 grams. For instance, a Jet cup attrition index of the combustion additive can be at least 5 times, at least 6 times, or even at least 7 times that of a Jet cup attrition index of the catalyst, where the Jet cup attrition is measured with 45 μm threshold after 6 hours at ambient temperature, 300 ft/s jet velocity, a flow rate of 98 L/min, and a sample loading of 100 grams. In embodiments, a Jet cup attrition index of the combustion additive is greater than a Jet cup attrition index of the catalyst, where the Jet cup attrition is measured with 20 μm threshold after 6 hours at ambient temperature, 300 ft/s jet velocity, a flow rate of 98 L/min, and a sample loading of 100 grams. In embodiments, a Jet cup attrition index of the combustion additive is at least 4 times that of a Jet cup attrition index of the catalyst, where the Jet cup attrition is measured with 20 μm threshold after 6 hours at ambient temperature, 300 ft/s jet velocity, a flow rate of 98 L/min, and a sample loading of 100 grams. For instance, a Jet cup attrition index of the combustion additive can be at least 5 times, at least 6 times, or even at least 7 times that of a Jet cup attrition index of the catalyst, where the Jet cup attrition is measured with 20 μm threshold after 6 hours at ambient temperature, 300 ft/s jet velocity, a flow rate of 98 L/min, and a sample loading of 100 grams. Without intending to be bound by any particular theory, it is believed that having a Jet cup attrition index of the combustion additive that is less than 4 times of a Jet cup attrition index of the catalyst can result in dilution and/or deactivation of the catalyst. On the contrary, it is believed that having a Jet cup attrition index of the combustion additive that is at least 4 times greater than a Jet cup attrition index of the catalyst can allow for improved selective removal of the combustion additive from the system. Thus, it is believed that this difference in attrition properties between the combustion additive and the catalyst can reduce a dilution effect of the catalyst as additional amounts of the combustion additive are added during operation of the reactor system. As used herein, the attrition index is measured using standard Jet Cup method of PSRI using 3″ Conical Jet Cup with orifice with inner diameter of 0.1875 inches.

In one or more embodiments, the amount of the combustion additive introduced to the reactor system 102 per addition is from 0.01 volume percent (vol. %) to 2 vol. % of a sum of a volume of the catalyst and a volume of the combustion additive. For example, the amount of the combustion additive introduced to the reactor system 102 may be from 0.01 vol. % to 1.5 vol. %, from 0.01 vol. % to 1 vol. %, from 0.01 vol. % to 0.5 vol. %, from 0.05 vol. % to 1.5 vol. %, from 0.05 vol. % to 1 vol. %, from 0.05 vol. % to 0.5 vol. %, from 0.5 vol. % to 2 vol. %, from 0.5 vol. % to 1.5 vol. %, from 0.5 vol. % to 1 vol. %, from 1 vol. % to 2 vol. %, from 1 vol. % to 1.5 vol. %, or from 1.5 vol. % to 2 vol. % of a sum of a volume of the catalyst and a volume of the combustion additive. In embodiments, the amount of the combustion additive to be introduced to the reactor system 102 can be modified based on the monitoring of the composition of the combustion gas in the combustor. Without intending to be bound by any particular theory, it is believed that adding the combustion additive with the catalyst in an amount greater than 2 vol. % can dilute the catalyst in the reactor system such that the catalytic dehydrogenation activity is reduced. On the contrary, it is believed that not adding the combustion additive with the catalyst when the system is deficient of combustion activity can result in a decrease of the fuel gas conversion.

It should be understood that, once introduced to the reactor system 102, the combustion additive will mix with the catalyst and, as a result, cycle through the reactor system 102 as discussed previously with regard to the catalyst. In other terms the introduction of the combustion additive to the reactor system 102 may produce a catalyst system that is a mixture of the catalyst and combustion additive. Due to the natural change in properties of the catalyst and combustion additive during operation of the reactor system 102, the properties and amounts of combustion additive and/or catalyst may refer to the properties and amounts of the combustion additive and/or catalyst upon introduction of the combustion additive to the reactor system 102.

### Test Methods

The various test methods of the present disclosure will be further discussed and referenced in the examples that follow.

### Propane Dehydrogenation Test

Propane dehydrogenation testing of samples that include catalyst and combustion additives without additional modification are carried out at ambient pressure in a fixed bed system under lab simulated reaction and regeneration cycles. The cycle includes a reaction step which is carried out under reactor temperature of 625° C., a reaction time of 60 seconds, a weight hourly space velocity (WHSV) of propane of 10 hr⁻¹, where the WHSV is defined as the weight of feed flowing per unit weight of the sample per hour, and a gas feed composition of 90 volume % (vol. %) propane and 10 vol. % N₂. After completing the reaction step, the sample undergoes a regeneration step where it is heated in air at 730° C. for 5 minutes. The reaction and regeneration cycle are repeated in rotation for a total of 10 cycles. For each reaction step, the reaction effluent at 15 seconds time on stream is analyzed using gas chromatography (GC). The feed conversion and product selectivity were determined by equations (1) and (2):

$$Feed Conversion = \frac{{\sum }_{k} {n}_{k} {C}_{k}}{{n}_{C 3} {C}_{C 3} + {\sum }_{k} {n}_{k} {C}_{k}} \qquad ( 1 )$$

in which:
   k refers to Product k, and the products analyzed include methane, ethane, ethylene, propylene, species including four carbon atoms (C4s), species including five carbon atoms (C5s), and species including six or more carbon atoms (C6s+);
   nₖ refers to the number of carbons in the chemical formula of Product k; n_{c3=} refers to the number of carbons in the specific product, propylene; while n_{C3} refers to the number of carbons in chemical formula of reactant propane; both n_{C3=} and n_{C3} are equal to 3; and
   Cₖ refers to molar fraction of general Product k in the reaction effluent, and C_{C3=} refers to the molar fraction of specific product, propylene, in the reaction effluent; while C_{C3} refers to the molar fraction of unreacted reactant propane, in the reaction effluent. The generation of coke during the conversion is not included in the feed conversion calculation because of the minimal contribution to the calculation (<0.5%).

### Approach to Equilibrium Conversion Calculation

The approach to equilibrium conversion (% ATE) can be calculated using equation 3. The approach to equilibrium conversion is the ratio of the feed conversion, as measured in equation 1 divided by the equilibrium conversion:

$$Product Selectivity = \frac{{n}_{C 3 =} {C}_{C 3 =}}{{\sum }_{k} {n}_{k} {C}_{k}} \qquad ( 2 )$$

in which the equilibrium conversion is calculated, as disclosed in D. Stull/The chemical thermodynamics of organic compounds (1969), published by J. Wiley, ISBN-10:9780471834908.

### Fuel Gas Combustion Evaluation

Fuel gas combustion testing of samples that include catalyst and combustion additives without additional modification is carried out in a fixed bed lab reactor at ambient pressure with a WHSV CH₄ of 0.59 hr⁻¹, where the WHSV CH₄ is defined as the weight of methane in feed flowing per unit weight of the sample per hour. The sample (0.1 g catalyst or combustion additive mixed with 0.1 g SiC diluent) is heated to 730° C. under a flow of N₂. Subsequently, N₂ is replaced by air and a flow of helium, where the helium constitutes 1.2 vol. % of the feed. After 20 min, methane is introduced until the feed gas contains 2 vol. % methane and 1.2 vol. % helium where the balance is synthetic air. These testing conditions are more severe than commercial operation to allow for greater differentiation of combustion performance. GC samples are taken continuously from an outlet and feed gas and analyzed to determine methane conversion. The methane conversion % is calculated using Equation 4 at 3 hours time on stream:

$$Approach to Equilibrium Conversion = \frac{Feed Conversion}{Equilibrium Conversion} \qquad ( 3 )$$

in which C refers to concentration of compound (CO, CO₂ or CH₄) in effluent stream.

### Jet Cup Attrition Test

Attrition evaluation of the catalyst support and combustion additive support is carried out using a 3 inch Jet cup attrition unit designed and fabricated by Particulate Solid Research, Inc (PSRI). The attrition index derived from the Jet cup attrition test is used to define the attrition of support materials. Because of the low metal loading of the catalyst and combustion additive, there is no difference (within testing error) in attrition index between support materials and the catalysts and combustion additives made therefrom. Thus the attrition of the catalyst support materials are representative of the attrition of the catalyst and combustion additive comprising the support materials described herein. The jet cup used in the tests is a 3 inch size conical shape jet cup with orifice with inner diameter of 0.1875 inches. The tests are carried out at ambient temperature, 300 ft/s jet velocity, a flow rate of 98 L/min, a sample loading of 100 grams, and 6 hours of attrition duration. The particle size distribution of materials before and after attrition test are characterized using laser diffraction technique (Beckman Coulter LS13 320). The absolute percentage of increase of particles (“fines”)<20 μm and <45 μm are reported as attrition index (A.I.) (20 μm) and A.I (45 μm), as shown in equations 5 and 6, respectively:

$$Methane Conversion = \frac{{C}_{CO} + {C}_{CO 2}}{{C}_{CH 4} + {C}_{CO} + {C}_{CO 2}} \qquad ( 4 )$$

### EXAMPLES

The various embodiments of the present disclosure will be further clarified by the following examples. The examples are illustrative in nature and should not be understood to limit the subject matter of the present disclosure

The catalyst and combustion additives in the Examples were prepared by conventional incipient wetness impregnation method to load a designated amount of metal to microspherical silica containing alumina supports with an average particle size ranging from 5 to 300 μm, pore volume of 0.35±0.20 mL/g, surface area of 100±50 m²/g, silica content of 2.5±2.5 wt %, and varying attrition properties. The support material with low attrition is referred as Base Support, while two materials, Support A and Support B, have higher attrition (See Example 4). The metal precursors used are nitrates or amine nitrates. The obtained materials were dried at temperature less than 200° C., and then calcined at temperature less than 800° C. Base Support was used for making the catalyst, while Support A and Support B were used for making the combustion additives.

### Example 1. Combustion Additives Comprising Manganese

The combustion additives of Example 1 included manganese and did not include platinum or gallium. Specific support material and composition for each sample is listed in Table 1. Propane conversion %, approach to equilibrium (ATE) conversion %, propylene selectivity %, and methane conversion % were measured according to the test methods described herein, and are reported in Table 1.

TABLE 1

| | | | Propane | ATE | Propylene | Methane |
|---|---|---|---|---|---|---|
| Example | | Mn, | Conversion | Conversion | Selectivity | Conversion |
| No. | Support | wt. % | % | % | % | % |
| Ex. 1-1 | Support A | 1.0 | 5.5 | 8.9 | 89.5 | 78.4 |
| Ex. 1-2 | Support B | 1.5 | 2.4 | 3.9 | 66.9 | 76.7 |
| Ex. 1-3 | Support A | 2.5 | 2.4 | 3.9 | 65.4 | 91.5 |

As seen in Table 1, the combustion additives of Example 1 exhibited improved combustion activity, as indicated by a higher methane conversion % compared to the catalysts of Comparative Example A. Further, increasing the wt. % of manganese in the combustion additive from 1 wt. % (Ex. 1-1) to 2.5 wt. % (Ex. 1-3) resulted in further improvement of the combustion activity of the combustion additive.

However, little dehydrogenation activity can be provided by these combustion additives and therefore a dilution effect may occur if such combustion additives build up in the catalyst system. The support materials selected for these additives (Support A and Support B) have higher attrition compared to that of the catalysts of Example A (Base Support) and Example B (Base Support), and therefore the combustion additives could be selectively removed in the fluid bed catalyst operation to reduce or eliminate the dilution effect.

### Example 2. Combustion Additives Comprising Iron

The combustion additives of Example 2 included iron and did not include platinum or gallium. Specific support material and composition for each sample is listed in Table 2. Propane conversion %, approach to equilibrium (ATE) conversion %, propylene selectivity %, and methane conversion % were measured according to the test methods described herein, and are reported in Table 2.

TABLE 2

| | | | Propane | ATE | Propylene | Methane |
|---|---|---|---|---|---|---|
| Example | | Fe, | Conversion | Conversion | Selectivity | Conversion |
| No. | Support | wt. % | % | % | % | (%) |
| Ex. 2-1 | Support A | 1.0 | 3.5 | 5.7 | 71.7 | 75.7 |
| Ex. 2-2 | Support B | 1.5 | 4.1 | 6.7 | 74.3 | 84.6 |
| Ex. 2-3 | Support A | 2.5 | 4.8 | 7.8 | 67.5 | 90.6 |

As seen in Table 2, the combustion additives of Example 2 exhibited improved combustion activity, as indicated by a higher methane conversion % compared to the catalysts of Comparative Example A. Further, increasing the wt. % of iron in the combustion additive from 1 wt. % (Ex. 2-1) to 2.5 wt. % (Ex. 2-3) resulted in further improvement of the combustion activity of the combustion additive.

Similar to the combustion additives of Example 1, the combustion additives of Example 2 provide reduced dehydrogenation activity. Therefore, high attrition support materials were selected for these additives (Support A and Support B) so they may be selectively removed in the fluid bed catalyst operation to reduce the dilution effect on dehydrogenation performance.

### Example 3. Combustion Additives Comprising Cobalt or Tin

The combustion additives of Example 3 included cobalt or tin and did not include platinum or gallium. Specific support material and composition for each sample is listed in Table 3. Propane conversion %, approach to equilibrium (ATE) conversion %, propylene selectivity %, and methane conversion % were measured according to the test methods described herein, and are reported in Table 3.

TABLE 3

| | | | Propane | ATE | Propylene | Methane |
|---|---|---|---|---|---|---|
| Example | | Metal, | Conversion | Conversion | Selectivity | Conversion |
| No. | Support | wt. % | % | % | % | (%) |
| Ex. 3-1 | Support | Co = 1.5 | 3.5 | 5.7 | 71.7 | 75.7 |
| | B | | | | | |
| Ex. 3-2 | Support | Sn = 1.5 | 4.1 | 6.7 | 74.3 | 84.6 |
| | B | | | | | |

As seen in Table 3, the combustion additives of Example 3 exhibited improved combustion activity, as indicated by a higher methane conversion % compared to the catalysts of Comparative Example A. Similar to the combustion additives of Example 1 and Example 2, the combustion additives of Example 3 provide reduced dehydrogenation activity. Therefore, high attrition support materials were selected for these additives (Support A and Support B) so they may be selectively removed in the fluid bed catalyst operation to reduce dilution effect on dehydrogenation performance.

### Comparative Example A. Catalysts Comprising Platinum and Gallium

The catalysts of Comparative Example A included platinum and gallium and did not include manganese. Specific support material and composition for each sample is listed in Table 4. Propane conversion %, approach to equilibrium (ATE) conversion %, propylene selectivity %, and methane conversion % were measured according to the test methods described herein, and are reported in Table 4. In embodiments, these catalysts can be used to provide good dehydrogenation performance.

TABLE 4

| | | | | Propane | ATE | Propylene | Methane |
|---|---|---|---|---|---|---|---|
| Example | | Pt, | Ga, | Conversion | Conversion | Selectivity | Conversion |
| No. | Support | ppmw | wt. % | % | % | % | % |
| Ex. A-1 | Base | 80 | 1.6 | 47.0 | 76.3 | 96.8 | 30.2 |
| | Support | | | | | | |
| Ex. A-2 | Base | 80 | 2.6 | 45.0 | 73.1 | 95.5 | 35.5 |
| | Support | | | | | | |

As seen in Table 4, the catalysts comprising platinum and gallium (Ex. A-1 and Ex. A-2) exhibit less combustion activity, as indicated by a lower methane conversion % compared to the combustion additives of Examples 1-3.

### Comparative Example B. Combustion Additives Comprising Manganese

The catalysts of Comparative Example B included manganese and did not include platinum or gallium. Specific support material and composition for each sample is listed in Table 5. Propane conversion %, approach to equilibrium (ATE) conversion %, propylene selectivity %, and methane conversion % were measured according to the test methods described herein, and are reported in Table 5.

TABLE 5

| | | | Propane | ATE | Propylene | Methane |
|---|---|---|---|---|---|---|
| Example | | Mn, | Conversion | Conversion | Selectivity | Conversion |
| No. | Support | wt. % | % | % | % | % |
| Ex. B-1 | Base | 0.15 | 11.0 | 17.9 | 94.5 | 25.3 |
| | Support | | | | | |
| Ex. B-2 | Base | 1.0 | 2.8 | 4.5 | 74.8 | 68.6 |
| | Support | | | | | |

As seen in Table 5, the catalyst comprising 0.15 wt. % manganese in the absence of platinum and gallium (Ex. B-1) exhibited low combustion activity, as indicated by a lower methane conversion % compared to the combustion additives of Example 1. Significant enhancement of the combustion performance is observed when increasing the manganese concentration to 1 wt. % (Ex. B-2). The dehydrogenation performance of the catalysts of Comparative Example B is low in the absence of platinum and gallium. However, contrary to the combustion additives of Examples 1-3, the support material selected for these catalysts (Base support) would have similar catalyst attrition rates to that of Example A. As the dehydrogenation performance of Comparative Example B is low, despite its enhanced combustion activity compared to Comparative Example A, a dilution effect may occur if such catalysts build up in the catalyst system.

### Example 4. Attrition Testing

The attrition of support materials was measured using the jet cup attrition test, as described herein. The results of the A.I. (20 μm) and A.I. (45 μm) of the support materials are shown in Table 6. The A.I. indicates the absolute difference in a percentage of fines having a particle size less than a certain threshold value before and after the jet cup attrition test, as described herein. For instance, an A.I. (20 μm) of 3.7 indicates that the absolute percentage of fines having a particle size <20 μm increased by 3.7% after the attrition test, while A.I. (45 μm) of 5.1 indicates that the raw percentage of fines having a particle size <45 μm increased by 5.1% after 6 hours of attrition duration for the Base Support. For Support A, the absolute percentage of fines having a particle size <20 μm increased by 23.9% after the attrition test, while the absolute percentage of fines having a particle size <45 μm increased by 39.9% under the same conditions as the Base Support attrition test.

TABLE 6

| | A.I. (20 μm) | A.I. (45 μm) |
|---|---|---|
| Base Support | 3.7 | 5.1 |
| Support A | 23.9 | 39.9 |
| Support B | 18.1 | 28.3 |

As shown in Table 6, the A. I. results indicate that the attrition rate of Support A or combustion additive is greater than 700% of the attrition rate of Base Support or catalyst under Standard PSRI testing described herein. In other words, the time in unit until mechanical attrition of the combustion additive comprising either Support A or Support B is expected to be less than that of the catalyst comprising base support in the reactor system.

A first aspect of the present disclosure is directed to a method for making light olefins by dehydrogenation comprising operating a catalytic dehydrogenation process, monitoring a composition of a combustion gas in the combustor to detect a concentration of one or more hydrocarbons, and selectively adding a combustion additive with the catalyst when the combustion gas comprises one or more hydrocarbons in an amount greater than 5% of a lower flammability level of the combustion gas at a temperature and pressure of the combustor. The operating comprises contacting a hydrocarbon-containing feed with a catalyst in a reactor to form an olefin-containing effluent, at least partially separating the olefin-containing effluent from the catalyst, passing the catalyst to a combustor and heating the catalyst by combusting a supplemental fuel, wherein the supplemental fuel comprises methane in an amount of greater than or equal to 1 mol. %, and passing the catalyst from the combustor to the reactor, such that at least a portion of the catalyst continuously cycles between the reactor and the combustor. A Jet cup attrition index of the combustion additive is greater than a Jet cup attrition index of the catalyst, where the Jet cup attrition is measured with 45 μm threshold after 6 hours at ambient temperature, 300 ft/s jet velocity, a flow rate of 98 L/min, and a sample loading of 100 grams. The combustion additive comprises from 1 wt. % to 10 wt. % of one or more transition metals exclusive of gallium and noble metals, from 0 parts per million by weight (ppmw) to 100 ppmw of gallium and noble metals, and at least 85 wt. % support.

A second aspect of the present disclosure may include the first aspect, wherein the one or more transition metals are chosen from manganese, iron, cobalt, tin, or combinations thereof.

A third aspect of the present disclosure may include either one of the second or third aspects, wherein the combustion additive comprises from 1 wt. % to 10 wt. % of manganese.

A fourth aspect of the present disclosure may include any one of the first through third aspects, wherein the combustion additive comprises from 1 wt. % to 10 wt. % of iron

A fifth aspect of the present disclosure may include any one of the first through fourth aspects, wherein the combustion additive comprises from 1 wt. % to 10 wt. % of cobalt.

A sixth aspect of the present disclosure may include any one of the first through fifth aspects, wherein the combustion additive comprises from 1 wt. % to 10 wt. % of tin.

A seventh aspect of the present disclosure may include any one of the first through sixth aspects, wherein the combustion additive further comprises from 0.01 wt. % to 5 wt. % of one or more alkali or alkaline earth metals.

An eighth aspect of the present disclosure may include any one of the first through seventh aspects, where the Jet cup attrition index of the combustion additive is at least 4 times that of the Jet cup attrition index of the catalyst.

A ninth aspect of the present disclosure may include any one of the first through eighth aspects, wherein a dehydrogenation catalytic activity of the combustion additive is less than 25 percent of a dehydrogenation catalytic activity of the catalyst.

A tenth aspect of the present disclosure may include any one of the first through ninth aspects, wherein the hydrocarbon-containing feed comprises one or more of ethane, propane, n-butane, or i-butane, and the olefin-containing effluent comprises one or more of ethylene, propylene, and butylene.

An eleventh aspect of the present disclosure may include any one of the first through tenth aspects, wherein the olefin-containing effluent comprises at least 20 wt. % light olefins.

A twelfth aspect of the present disclosure may include any one of the first through eleventh aspects, wherein the hydrocarbon-containing feed comprises propane and the olefin-containing effluent comprises propylene.

A thirteenth aspect of the present disclosure may include any one of the first through twelfth aspects, wherein the supplemental fuel further comprises natural gas, ethane, propane, hydrogen, or combinations of two or more thereof.

A fourteenth aspect of the present disclosure may include any one of the first through thirteenth aspects, wherein the catalyst comprises from 0.05 wt. % to 10 wt. % of one or more metals chosen from gallium, indium, thallium, or combinations thereof, from 5 ppmw to 1000 ppmw of one or more metals chosen from platinum, palladium, rhodium, iridium, ruthenium, osmium, or combinations thereof, and at least 85 wt. % support.

A fifteenth aspect of the present disclosure may include any one of the first through fourteenth aspects, wherein the catalyst comprises from 0.05 wt. % to 10 wt. % gallium, from 5 ppmw to 1000 ppmw of platinum, and at least 85 wt. % support.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

1. A method for making light olefins by dehydrogenation, the method comprising:
operating a catalytic dehydrogenation process, the operating comprising:
contacting a hydrocarbon-containing feed with a catalyst in a reactor to form an olefin-containing effluent;
at least partially separating the olefin-containing effluent from the catalyst;
passing the catalyst to a combustor and heating the catalyst by combusting a supplemental fuel, wherein the supplemental fuel comprises methane in an amount of greater than or equal to 1 mol. %;
passing the catalyst from the combustor to the reactor, such that at least a portion of the catalyst continuously cycles between the reactor and the combustor;
monitoring a composition of a combustion gas in the combustor to detect a concentration of one or more hydrocarbons; and
selectively adding a combustion additive with the catalyst when the combustion gas comprises one or more hydrocarbons in an amount greater than 5% of a lower flammability level of the combustion gas at a temperature and pressure of the combustor, wherein a Jet cup attrition index of the combustion additive is greater than a Jet cup attrition index of the catalyst, where the Jet cup attrition is measured with 45 μm threshold after 6 hours at ambient temperature, 300 ft/s jet velocity, a flow rate of 98 L/min, and a sample loading of 100 grams, and wherein the combustion additive comprises:
from 1 wt. % to 10 wt. % of one or more of tin or transition metals exclusive of gallium and noble metals;
from 0 parts per million by weight (ppmw) to 100 ppmw of gallium and noble metals; and
at least 85 wt. % support.

2. The method of claim 1, wherein the one or more transition metals are chosen from manganese, iron, cobalt, or combinations thereof.

3. The method of claim 1, wherein the combustion additive comprises from 1 wt. % to 10 wt. % of manganese.

4. The method of claim 1, wherein the combustion additive comprises from 1 wt. % to 10 wt. % of iron.

5. The method of claim 1, wherein the combustion additive comprises from 1 wt. % to 10 wt. % of cobalt.

6. The method of claim 1, wherein the combustion additive comprises from 1 wt. % to 10 wt. % of tin.

7. The method of claim 1, wherein the combustion additive further comprises from 0.01 wt. % to 5 wt. % of one or more alkali or alkaline earth metals.

8. The method of claim 1, wherein the Jet cup attrition index of the combustion additive is at least 4 times that of the Jet cup attrition index of the catalyst.

9. The method of claim 1, wherein a dehydrogenation catalytic activity of the combustion additive is less than 25 percent of a dehydrogenation catalytic activity of the catalyst.

10. The method of claim 1, wherein the hydrocarbon-containing feed comprises one or more of ethane, propane, n-butane, or i-butane, and the olefin-containing effluent comprises one or more of ethylene, propylene, and butylene.

11. The method of claim 1, wherein the olefin-containing effluent comprises at least 20 wt. % light olefins.

12. The method of claim 1, wherein the hydrocarbon-containing feed comprises propane and the olefin-containing effluent comprises propylene.

13. The method of claim 1, wherein the supplemental fuel further comprises natural gas, ethane, propane, hydrogen, or combinations of two or more thereof.

14. The method of claim 1, wherein the catalyst comprises:
from 0.05 wt. % to 10 wt. % of one or more metals chosen from gallium, indium, thallium, or combinations thereof;
from 5 ppmw to 1000 ppmw of one or more metals chosen from platinum, palladium, rhodium, iridium, ruthenium, osmium, or combinations thereof; and
at least 85 wt. % support.

15. The method of claim 1, wherein the catalyst comprises:
from 0.05 wt. % to 10 wt. % of gallium;
from 5 ppmw to 1000 ppmw of platinum; and
at least 85 wt. % support.
